(19) United States
(12) Patent Application Publication
(10) Pub. No.: US 2007/0264670 A1
(43) Pub. Date: Nov. 15, 2007

(54) Method for Screening and Selecting Enzymes

(75) Inventors: Albertus Alard Van Dijk, Vlaardingen (NL); Marcellis Wilhelmus Elisabeth Maria Van Tilborg, Leiden (NL); Jort Steven Johan Gerritsma, Leiden (NL); Maurien Martha Agatha Olsthoorn, Utrecht (NL); Baukje Folkertsma, Delft (NL); Ilja Westerlaken, Rotterdam (NL)

Correspondence Address: NIXON & VANDERHYE, PC, 901 NORTH GLEBE ROAD, 11TH FLOOR, ARLINGTON, VA, 22203, US

(73) Assignee: DSM IP Assets B.V., Heerlen (NL)

(21) Appl. No.: 11/631,011

(22) PCT Filed: Jun. 30, 2005
(86) PCT No.: PCT/EP05/53106

§ 371 Date: Dec. 28, 2006

(30) Foreign Application Priority Data
Jul. 1, 2004 (EP) 04103109.7
Dec. 23, 2004 (EP) 04106948.5

(51) Int. Cl. G01N 33/573; C12P 1/04

(52) U.S. Cl. 435/7.4; 435/69.1; 435/71.1

(57) ABSTRACT

The present invention relates to a method for identifying an enzyme of interest comprising screening a set of candidate enzymes on an application matrix, preferably on at least a medium throughput level, and selecting enzymes for their ability to produce a functionality change in the application matrix. In the application matrix screening method of the invention it is not necessary to define the enzyme activity to be screened prior to applying the screening method.

Current strategies for new enzyme product development are hypothesis driven. Firstly, the required enzyme activity for a particular application is defined, followed by a search for the relevant enzyme using as screening tool an enzyme activity assay dedicated to the enzyme activity as defined. The search typically includes the screening of one or more organisms that could express the desired enzyme activity. Often these organisms are microorganisms such as bacteria, yeasts and fungi.

Alternatively, the relevant enzyme may be identified by searching sequence databases for genes that encode or are expected to encode a protein that has the desired enzyme activity. These searches rely on the assignment of a function to a new gene based on homology of the encoded gene product with already known gene products (annotation). When a gene of interest has been identified it can be cloned and expressed using techniques known in the art, and the protein that is produced can be tested for the desired activity.

These approaches in the end lead to the identification of one or more enzymes that have the desired activity. Once these enzymes are identified they are tested in final application for proper functionality. This approach, and especially the route via the database search, benefits from the enormous and still increasing pool of annotated genes that becomes available from current sequencing programmes.

However, the hypothesis driven approach also has several drawbacks. For instance, the testing of a relevant enzyme in final application only occurs in the final stage of the screening, implying that the success of an often labour-intensive series of actions including cultivation of a large set of microorganisms is only verified at the end of the screening. Often it turns out that the working hypothesis was wrong, leading to a search for the wrong enzyme activity and failure of the screening effort.

It further often occurs that the relevant enzyme activity for a particular final application cannot be defined at all in advance, because of lack of understanding of the application system. In such cases, a screening assay cannot be defined as well and a search for the relevant enzyme is not possible.

In case annotated genes are used as a source to identify new enzymes, there is a strong dependence on quality of the annotation. It may occur that the annotation is wrong. In addition, the use of annotated genes is an inherently conservative approach that will not lead to the identification of entirely new and unexpected gene products.

Although a (probable) function can now be assigned to a lot of genes, there is still a considerable amount of genes that cannot be annotated. Percentages of not-annotated genes vary between organisms but may range from 20-70%. This means that a considerable number of genes is not included in the search for relevant enzymes in the database approach and thus is not exploited in hypothesis based screening.

In hypothesis based screening, an enzyme assay is always required to identify the relevant enzyme. Assay development is a complicated matter and in fact often forms a bottleneck in enzyme screening. Assay development often includes the synthesis of suitable substrates that can be used to identify the relevant enzyme. Synthesis of the proper substrates is often a major hurdle in assay development. Alternatively, the natural substrate for the enzyme may be chemically or enzymatically modified such that it becomes a suitable substrate for screening purposes (e.g. the attachment of dye compounds to the natural substrate). However, if the synthesized substrate is not identical to the natural substrate, the identified enzymes that show activity on the artificial substrate may not show activity on the natural substrate.

In order to circumvent the drawbacks of the hypothesis driven approach, the present invention provides a complementary form of screening: application matrix screening.

In application matrix screening according to the present invention, the screening parameter is a functionality change in the application matrix.

In the application matrix screening according to the invention, the enzyme activity does not need to be defined in advance, i.e. does not need to be known prior to the screening process. This is a principle difference with hypothesis based screening, in which firstly the enzyme activity is defined that should produce a desired defined change in matrix functionality, whereupon a suitable substrate is chosen or prepared that is able to detect the prior defined enzyme activity.

International application WO 00/67025 discloses an example of such a hypothesis-based approach: it discloses the preparation of synthetic fluorescence-marked acylglyceride substrates that can be used in the screening for lipases. Thus, these substrates are deliberately developed for the screening of a defined enzyme, i.e. lipase, activity and additionally do not resemble the final application wherein the lipase is to be used. Contrary, the application matrix screening according to the invention is not limited in any way by a need for prior definition of an enzyme activity and therefore allows the identification of enzymes that would not have been identified as being relevant in the hypothesis based approach. The approach according to the invention will substantially increase the hit-rate of enzyme screening.

For example, when an expression library is screened for a particular functionality change in an application matrix, e.g. reduction of viscosity in a soy meal slurry, all clones that code for an enzyme that can accomplish this functionality change are identified in the screening process. Contrary, in the hypothesis based screening firstly the enzyme activities that could accomplish this change are defined, e.g. a protease or one or a particular group of cell wall degrading enzymes (or a combination thereof), whereupon an enzyme assay is developed directed to finding such an enzyme in a screening.

In a first aspect, the present invention therefore provides a method for identifying an enzyme of interest, wherein a set of candidate enzymes is screened on an application matrix, preferably on at least a medium throughput level, and enzymes are selected for their ability to cause a functionality change in the application matrix.

In the context of the present invention, a functionality change in an application matrix is defined as any physical or chemical change in the application matrix resulting from an enzymatic reaction that can be measured or detected by any analytical means. Examples of such a functionality change are a change in aggregation state, colour, water binding, viscosity, gel-formation, flavour profile, odour formation, etc.

The relevant enzyme activity may be at least one of but is not limited to those exerted by proteases, lipases, cellulases, oxidoreductases, transferases, isomerases, ligases, esterases, hydrolases, carbohydrases, mono-oxygenases, di-oxygenases.

In particular, the method of the invention comprises the following steps:

a) providing a set of candidate enzymes,

b) screening said set of candidate enzymes on an application matrix, preferably on at least a medium throughput level,

c) selecting a subset of enzymes that produces a functionality change in the application matrix,

d) selecting an enzyme from the subset of enzymes with a desired activity in a further application test,

e) defining the enzyme activity (ies) of the enzyme(s).

Defining the enzyme activity (ies) of the enzyme(s) may be done at any stage of the process of the invention. However, there is no need for a prior accommodation of the application matrix with respect to the nature of the enzyme activity (ies) to be screened.

The set of candidate enzymes can be generated in several ways. For instance, the set of candidate enzymes is provided by a set of microorganisms cultivated under conditions conducive to the expression of one or more enzymes. The members of the set of microorganisms may be different microbial species and/or may be different strains from one particular microbial species. The one or more enzymes may accumulate intracellularly or may be secreted into the culture medium.

To subject candidate enzymes to the screening according to the invention, individual clones (within an expression library) are cultivated until a desired biomass concentration or desired cell density is reached. Depending on whether the candidate enzymes are produced intracellularly or secreted into the culture medium, cell extracts or culture supernatants, respectively, are prepared for screening.

For screening of intracellularly accumulating enzymes, cells need to be treated to release the intracellular enzymes for interaction with the application matrix in the screening according to the invention. Typically, cell-free extracts are prepared according to methods known to those skilled in the art. For screening of extracellular enzymes, cells are removed from the culture liquid by centrifugation, optionally followed by an ultrafiltration step, whereupon the culture supernatant is subjected to screening.

Preferably, the set of candidate enzymes is provided by a set of corresponding genes or cDNAs cloned and expressed in a suitable host organism, i.e. is provided by an expression library. The choice for the source of the set of genes or cDNAs encoding the set of candidate enzymes and/or for the host organism will depend among others on the intended application of the enzyme to be selected.

The set of genes or cDNAs encoding the set of candidate enzymes may originate from any organism of interest, i.e. an animal (including human) or plant species or a microorganism. Expression libraries can be made in various host organisms such as bacteria (e.g. Escherichia coli, Bacillus subtilis), yeasts (e.g. Pichia pastoris, Saccharomyces cerevisiae, Kluyveromyces lactis), filamentous fungi (e.g Aspergillus niger, Aspergillus oryzae), insect cells or other protein expression systems known in the art such as cell free expression systems.

A set of genes or cDNAs originating from a bacterium preferably is expressed in Bacillus subtilis or Escherichia coli, a set of genes or cDNAs originating from a yeast in Pichia pastoris, Saccharomyces cerevisiae or Kluyveromyces lactis, a set of genes or cDNAs originating from a fungus in e.g. Aspergillus niger or Aspergillus oryzae, a set of genes or cDNAs originating from an animal or plant in a suitable microbial host (the choice of the host may for instance depend on the size and nature of the encoded enzyme).

Especially for organisms with a large genome, e.g. eukaryotes, it is preferred to use cDNAs to provide a set of candidate enzymes. It is an option to prepare and combine different cDNA libraries coming from organisms that are cultivated under different conditions.

The amount of clones to be screened for one hit depends among others on the source organism of the set of genes.

In the case of prokaryotes, expression libraries can be constructed from the mRNA population that is obtained under specific circumstances. By growing bacteria under various culturing conditions and sampling on various points in time, a rather full spectrum of mRNAs can be obtained. Methods to do this are well known in the art. Alternatively, methods have been described to construct a genomic bacterial (expression) library by cleaving the genomic DNA with specific endo-nucleases and cloning the obtained DNA fragments in a suitable vector. These approaches lead to the construction of bacterial expression libraries.

In the case of eukaryotes (filamentous fungi, yeasts, plants, etc), expression libraries are usually constructed in the form of cDNA libraries. Methods to construct such cDNA expression libraries are known in the art. Since the number of genes in eukaryotes (ranging from ˜6,000 for baker's yeast to ˜30,000 or more for plants) is much higher than that in bacteria (ranging from ˜2,500 to ˜4,500) and because the number of growth conditions required to obtain a full representation of the genome of a eukaryote is much higher than that of a prokaryotes, the library size (number of clones) of an expression library that gives a full representation of a eukaryotic genome is often a factor 10-100 larger than that required for a prokaryote. As a consequence, the number of clones from an expression library that needs to be screened to identify a particular gene is usually much larger for eukaryotes than for prokaryotes.

The method of library construction via cDNA's inherently implies a high redundancy in the expression bank: a lot of genes will be represented more than one time. This redundancy will increase considerably as the representation of all genes in the library reaches completeness. Therefore, in one embodiment of the invention, the set of candidate enzymes, e.g. the expression library, may be refined to decrease the number of candidates, yielding a refined set of candidate enzymes, also called a smart set of candidate enzymes. e.g. a smart expression library.

The refining process may remove the majority of clones that are represented more than once in the library, i.e. may remove redundant clones. As a result, only one or a few representative(s) of each clone is (are) left in the refined library, i.e. in the refined set of candidate enzymes. The refined set of candidate enzymes will therefore contain the minimal number of members that are considered relevant for the screening purpose. For strategic reasons, redundancy may be re-introduced in the expression library on purpose, e.g. to minimize failure in growth of a particular clone during cultivation of the entire expression library.

The refining process may also remove clones that have no or no significant protein over-expression, from the expression library. As a result, the refined expression library used for the screening according to the invention is enriched in clones that are protein over-producers. Protein over-producers are transformed clones that express protein from a cloned gene at such a level that the protein expression profile of the clone is distinguishable from the background protein expression profile of the untransformed expression host organism.

The refining process according to the invention, i.e. the screening of members of e.g. an expression library for redundancy and/or protein over-expression, can be done in various ways.

Protein over-expression can for instance be recognized as the appearance of one or more extra bands on SDS-PAGE. However, SDS-PAGE is laborious and 96-wells SDS-PAGE has a low resolution. SDS-PAGE analogues such as lab-on-a-chip systems, for instance those marketed by Agilent, are more suitable for at least medium throughput screening.

A system that appeared to be well suited for medium or even high throughput screening of members of an expression library is mass spectrometry. The refining process may be done on basis of the unique fingerprint that is obtained after, for instance, proteolytic digestion and mass spectrometric analysis of the clones of the library in question. Such an analysis can be done on extracellular (secreted) as well as on intracellular protein.

A typical procedure for analysis of extracellular protein would be as follows. First cells are removed from the culture liquid by centrifugation, optionally followed by an ultrafiltration step and/or a sample pre-treatment suitable to remove by-products, for instance carbohydrates and salts, and the cell-free supernatant is buffered to optimise conditions for proteolytic digestion. The sample is treated by the appropriate protease(s), e.g. trypsin, optionally followed by a (chromatographic) desalting, and injected into the mass spectrometer. The resulting mass spectrum consists of a profile of peptides with a molecular weight distribution that depends on the protein composition of the clone. Similar clones will give similar profiles, allowing identification of redundant clones. When a clone over-expresses a particular protein, this will result in extra peptides in the mass spectrum, compared to the clones that do not contain an over-expressed protein. The molecular weights of the extra peptides together with their fragmentation spectra are a unique fingerprint of the protein that is overproduced.

For analysis of intracellular protein, cell-free extracts may be prepared following techniques known to those skilled in the art. Preferably, extracts are prepared using a method that interferes as less as possible with subsequent screening steps.

A large variety of mass spectrometers is currently available, which differ in ionisation methodology (e.g. electro-spray ionisation (ESI) or matrix assisted laser desorption ionisation (MALDI)) and ways of mass determination (e.g. time of flight (TOF), quadrupole (Q), triple quadrupole (TQ) or quadrupole—time of flight (QTOF), ion cyclotron resonance (ICR), (linear) ion trap, etc). Preferably, a mass spectrometer with high resolution and duty cycle is used. In one set-up, the mass spectrometer of choice operates with MALDI as ionisation technology, and either a TOF, a QTOF, a double or triple Q or (linear) ion trap mass analyser.

The refining process described above is applicable to any expression library. Thus, the present invention provides a process to refine an expression library by removing redundancy and/or by removing clones that have no or no significant protein over-expression.

The unrefined or preferably the refined set of candidate enzymes is screened on a suitable application matrix and a subset of enzymes is selected that produces a functionality change in the application matrix. A functionality change can be any physical or chemical change in the application matrix resulting from an enzymatic reaction that can be measured or detected by any analytical means.

A suitable application matrix for use in the screening according to the invention can in principle be any matrix that resembles a particular final application of interest. It is thereby not necessary to deliberately accommodate the application matrix for screening a specific, predefined enzyme activity. Preferably, the final application of interest is a food and/or feed application.

Thus, the choice of the application matrix that is used for screening is determined among others by the desired final application wherein the enzyme to be selected should be active.

Preferably, the application matrix should be as close as possible to the final (large scale) application, i.e. should be of substantially the same composition as the final application substrate and/or should at least be a proven representative of the final application.

The skilled person will understand that the application matrix may for instance contain an amount of e.g. proteins, carbohydrates and/or lipids that slightly deviates from the amount present in the corresponding final application without deviating form the spirit of the invention. For instance, it may be necessary to use an application matrix that contains a higher water content than the final application. Alternatively or additionally, the composition of the application matrix, for instance relating to the protein, carbohydrate and/or lipid content or ratio, may slightly differ from the composition of the final application.

Preferably, the composition of the application matrix corresponds to the composition of the final application for at least 70 weight % (based on dry matter content of the application matrix as well as of the final application), more preferably for at least 80%, even more preferably for at least 90%, most preferably for at least 95%.

The application matrix should further preferably be produced reproducible over time. Reproducible production over time means that production of the application matrix should lead to a matrix with substantially the same overall composition over time and/or that production of the application matrix should not be subject to seasonal variations that cannot be accommodated. The application matrix should further preferably be compatible with screening on at least a medium throughput level (e.g. micro-titre formats) and preferably be compatible with pipetting and pumping procedures (e.g. be free of big lumps). The application matrix should preferably have a stable physical state.

For example, for bread making the application matrix can be a particular dough from which the bread is made or can be a proven representative of the dough from which the bread is made, e.g. a wheat (or other grain of interest) flour suspension or meal slurry; for cheese making the application matrix can be full fat (cows) milk or curd; for soy de-viscosifying the application matrix can be a concentrated soybean meal slurry.

Screening on a medium throughput level typically means that at least one thousand, such as one thousand to 100 thousand, samples (clones) are screened per week. Of course, the screening according to the invention may also suitably be done on a low throughput level, i.e. less than one thousand samples per week.

The present invention envisages various embodiments of the screening of a set of candidate enzymes on an application matrix of interest and subsequent selection of a subset of enzymes that produce a functionality change in the application matrix.

In one embodiment of the invention, a subset of enzymes is selected that produces a generic functionality change in the application matrix. A generic functionality change is a functionality change that is not defined in advance and that is not limited to a change of a particular nature. It may be any physical or chemical change in the application matrix resulting from an enzymatic reaction that can be measured or detected by any analytical means. It is thereby not necessary to define what type of functionality change the enzyme to be selected would achieve in the application matrix of interest.

Selection of a subset of enzymes producing a generic functionality change in the application matrix requires a very generic detection methodology and/or technology, i.e. methodology and/or technology that is capable of detecting any functionality change occurring in the application matrix. Several methods and/or technologies may be used for this purpose, such as those mentioned below.

Heat detection, e.g. micro calorimetric analysis, is in principle a suited technology, since essentially all (chemical) reactions are accompanied by changes in heat production (exothermic or endothermic). A drawback of heat detection technology is that it may not always be sensitive enough.

Another parameter that can be used to identify a generic functionality change in an application matrix is the dielectric constant of the matrix. Dielectric probes are available that very sensitively measure changes in dielectric constant of a medium, e.g. an application matrix.

Technologies based on semiconductor measurements, such as an electronic nose and/or mouth, are especially suitable in those cases where small molecules are generated.

Suitable technologies further are technologies based on infrared (IR) and RAMAN spectroscopy, such as Fourier Transform InfraRed Spectroscopy (FTIR). This is a technique, which detects vibrational transitions inflicted by an infrared beam. The frequency of the absorption bands in the spectra correlates with the energy difference between the vibrational ground state and excited state. These absorption bands are characteristic for particular groups of atoms. The spectra generated by FTIR can change due to generic functionality changes in an application matrix.

A very suitable technology for measuring generic changes in an application matrix is High Resolution Ultrasonic Spectroscopy (HRUS). High Resolution Ultrasonic Spectroscopy measurements are based on measurement of velocity and attenuation of acoustical waves at high, ultrasonic frequencies, propagating through the application matrix. The HRUS provides fast, non-destructive analysis of a wide spectrum of properties of matrices. The advantage of this technique is the ability of ultrasonic waves to propagate through optically nontransparent application matrices. The HRUS generates ultrasonic waves of longitudinal deformations using piezo electro transducers. The main characteristics determining the propagation of these waves are ultrasonic velocity and ultrasonic attenuation coefficients. The value of ultrasonic velocity is a function of density and the elastic modules of the longitudinal modulations. The absorption and the scattering of ultrasonic waves determine the value of the ultrasonic attenuation coefficient.

Summarizing, the HRUS technology is based on measuring the changes that take place to ultrasonic waves as they pass through an application matrix. Therefore ultrasonic measurement represents a preferred technology for the detection of generic functionality changes in an application matrix.

Therefore, in a further aspect, the present invention provides the use of ultrasonic spectroscopy, preferably HRUS, in the screening for enzyme activities. Specifically, a set of candidate enzymes as described above is screened on a suitable substrate and enzymes are selected for their ability to cause a functionality change in the substrate, wherein the functionality change is measured using ultrasonic spectroscopy, preferably HRUS. The suitable substrate can be any substrate for which it is desired to find an enzyme with an activity thereon. Preferably the suitable substrate is an application matrix as described above. Preferably, ultrasonic spectroscopy is done kinetically, as described herewith below.

In a preferred embodiment, the detection of a (generic) functionality change is done using a so-called kinetic analysis, i.e. an analysis wherein a reaction is followed in time. The time period over which the reaction is followed may vary from a few minutes, e.g. 2 or 5 or 10 minutes, to a couple of hours, e.g. 2 or 4 or 8, to 24 hours. Preferably, the reaction is followed starting from the beginning, i.e. from the moment the enzyme is added to the application matrix (T=0). Kinetic analysis advantageously allows a proper determination of the enzyme activity that is screened for versus background activity that could inherently be present in members of the set of candidate enzymes and/or in the application matrix itself.

In one embodiment of the invention, the result of the first screening and selection for a generic functionality change in the application matrix, as described above, is in fact a matrix-specific subset of the set of candidate enzymes, e.g. the expression library. Such a matrix-specific expression library advantageously only has to be generated once for each organism for a particular application matrix. The expression libraries can be stored so that they are directly available in case a new application question comes up.

In another embodiment of the invention, the subset of enzymes obtained after selecting those enzymes providing a generic functionality change in the application matrix, e.g. the matrix-specific expression library, is again screened on the application matrix and a further subset of enzymes is selected that produces a defined functionality change in the application matrix, i.e. a functionality change more resembling and/or closer to the desired functionality change in the final application.

A defined functionality change in the application matrix is defined in advance, depending on the functionality change that is desirable in the final application. It may for instance be one functionality change selected from a change in aggregation state, colour, water binding, viscosity, gel-formation, flavour profile, odour formation, etc.

The type of technique used for detection of a defined functionality change depends on which functionality change is defined. Defined functionality changes in the application matrix may be detected by the use of several measurements and/or combinations thereof, such as pH measurements, various calorimetric and spectroscopic techniques, use of thermistors, dielectric property measurements, ultrasound measurements, mass spectrometric measurements, dielectric constant determination, viscosity measurements, rheological measurements, etcetera.

Detection of these changes, relative to the untreated matrix, may take place by directly measuring the matrix or extracts thereof. Direct measurement of the matrix may e.g. be done via rheological measurements, reflection or absorbance measurements, viscosity measurements, etc.

Indirect measurements, i.e. measurements that are not performed on the matrix itself, may sometimes be required, e.g. in case release of low molecular weight components is measured from an insoluble matrix. In such a case the low molecular weight components could be extracted from the matrix to allow quantification. Alternatively, indirect measurements could include determination of a change in the composition of the volatile components (e.g. using GC-MS) released from the matrix upon enzymatic treatment.

In another embodiment of the invention, the set of candidate enzymes is screened on the application matrix and directly selected for the ability to produce a defined functionality change in the application matrix. A first screening for a generic functionality change in the application matrix is omitted.

In still another embodiment, the screening of the (sub)set of candidate enzymes for the ability to produce a defined functionality change in the application matrix is repeated at least once. In this embodiment, a first screening for a defined change in the application matrix may be for a less stringent change than a further screening. Upon the first screening, a crude selection is made in which the conditions for selection are not very stringent. The candidates selected in this step can then be further selected with a more stringent selection criterion. Such a two-stage selection may result in a higher throughput screening, and could therefore be desirable. An example of an application where this is two-stage selection may be relevant is the generation of flavour in cheese via specific amino acids. In the first stage, a set of candidate enzymes is selected that produces an increased level of free amino acids using e.g. detection by ninhydrin staining. Selected candidates would then be tested in a further screening for the generation of specific free amino acids. For cheese application, these specific amino acids could be e.g. methionine or phenylalanine being important flavour precursors in many cheese varieties.

The present invention advantageously allows that the enzyme activity (ies) associated with one or more enzyme molecule(s) may be defined at any stage of the process of the invention without necessitating a prior accommodation of the application matrix with respect to the nature of the enzyme activity (ies) to be identified.

Defining the enzyme activity (ies) of an enzyme may be done in various ways known to the person skilled in the art. For instance, it may be established which products are formed in the application matrix as a result of enzymatic activity of the (selected) enzyme. It may also be established on which particular (model) substrate(s) the selected enzyme shows activity. It is also possible to determine the amino acid sequence of the selected enzyme and analyse the sequence for homology with known sequences. The latter possibility may already be done at the stage of refining the expression library.

The screening on the application,matrix generally will take place on microtiterplate to test tube scale (100 μl to 5 ml). For sample incubation and transport, pipetting devices can be used, optionally in combination with robots and flow through systems. The choice of sample handling is greatly determined by the physical parameters of the application matrix involved.

The subset or further subset of enzymes obtained after screening a set of candidate enzymes on the application matrix is screened in a further application test at a larger scale. Although not necessarily full scale, the further application screening is generally at much larger scale (tens to hundreds of grams) than the application matrix screening which is at gram scale or lower. Examples of such further application tests are mini cheeses and puppy loafs. Candidates that show positive performance in this further application screening are selected and tested in full-scale application tests representing the final application.

Generally, the scale at which the screening is performed throughout the process of the invention increases. In return, the throughput capabilities typically decrease with the increase of scale because of logistical and handling reasons. Expression libraries are generally cultivated in quantities of a few to several hundreds or microliters, and analyses are at the same scale. Throughput at this level is in the order of at least thousand, such as thousand to 100's of thousand samples (clones) per week, which is medium throughput. Upon being subjected to the process of the invention, expression libraries are generally reduced in size to an estimated 20-30% of the total number of genes present in the genome of the organism from which the expression library is prepared. This implies that the number of relevant candidates is reduced to a few hundred to a few thousand, the precise number depending on the organism in question. The method of the invention advantageously allows a maximum of a few hundred candidates to being subjected to screening in a further application test. After screening in the further application test, a maximum of 10-20 candidates is generally left for full scale application testing. In total, this leads to the establishment of an effective selection funnel between expression library and full-scale application trials, wherein proteins are selected only on effectiveness in a particular application. Enzymes identified (selected) using the method of the invention advantageously have a high chance of proper and effective performance in full-scale application.

In full-scale application, substantial amounts of enzymes and substrate materials are usually required. Also, the costs involved at this scale are often considerable, limiting the throughput of this phase. The prior screening on the application matrix according to the invention advantageously reduces the number of enzymes that is to be tested on full-scale application.

The method of the invention is advantageously used to identify novel enzyme activities that would not be identifiable by hypothesis based screening methods.

The method of the invention can be used to select or identify one enzyme activity, but also to select or identify a mixture of two or more enzyme activities. Usually, a mixture of enzyme activities is provided by different enzyme molecules. However, it is also possible that a single enzyme molecule may provide different types of activities.

The present invention shows that a functionality change in an application matrix can advantageously be used as a screening parameter to select desired enzyme activities.

In a second aspect, the present invention relates to a process for the production of an enzyme selected by the method of the first aspect. The process comprises growing a microorganism capable of expressing said selected enzyme under conditions conducive to production of said enzyme and recovering the produced enzyme. Recovery of the enzyme may include steps like separation of microbial cells to produce a cell-free culture supernatant, concentration of the cell free supernatant by e.g. ultrafiltration, stabilisation of the liquid culture supernatant or ultrafiltrate by suitable stabilising agents, further purification of the enzyme from the liquid culture supernatant or ultrafiltrate, preparation of solid enzyme compositions by granulation and/or drying techniques. Preferably, the microorganism capable of expressing said selected enzyme is a host organism transformed with a polynucleotide containing an expression cassette encoding the selected enzyme.

### BRIEF DESCRIPTION OF THE FIGURES

FIG. 1. Ultrasound measurements of the activity of three enzymes in a milk matrix

FIG. 2. Ultrasound measurements of the activity of lipase in a cheese curd matrix

FIG. 3. Ultrasound measurements of the activity of five enzymes in wheat flour suspensions

FIG. 4. Ultrasound measurements of activity of pectinases in a soy-WUS matrix

FIG. 5. Ultrasound measurements of enzyme activity spiked in a culture supernatant and a cell-free extract

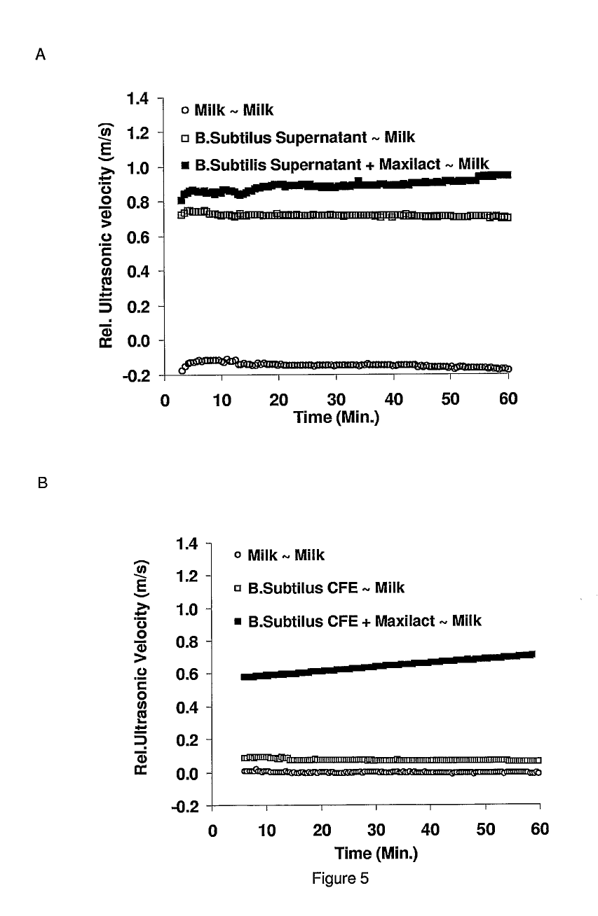

### EXAMPLE 1

### Preparation of a Cheese Curd Matrix

A representative way to prepare a cheese curd matrix is as follows. Full fat cows milk (250 grams) was heated in a centrifuge tube to 30° C. At this temperature and under gentle stirring, 165 μl of CaCl₂ was added directly followed by 54.4 μl Maxiren® 180 (obtained from DSM Food Specialties, The Netherlands). Stirring was then stopped and the mixture was left at 30° C. for 60 minutes. The centrifuge tube was then centrifuged in a Sorvall GS-3 rotor (5500 rpm, 25° C.) and the supernatant was carefully discarded. Cutters of stretched wire, spaced 1 cm apart on a frame, were used for manual cutting of the pellet. The volume of the pellet was measured, and an equal volume of a NaCl/Lactic acid solution (NaCl 90 g/L, 12.75 ml lactic acid/L) was added. The mixture was homogenized using an Ultra-Turrax. During homogenisation, 63 μl of cell lysate, prepared as described below, was added. The total volume was measured and the homogenate was diluted by addition of an amount of NaCl-solution (4.5% w/v) equal to 33% of this volume. The cheese curd matrix is now ready for use in application screening.

Preparation of the cell lysate:

The cell lysate was prepared as follows; 45 ml physiological salt solution was added to 5 ml of DELVO-TEC® LL55D (obtained from DSM Food Specialties, The Netherlands) cell suspension and centrifuged (5000 rpm, Sorvall GS3, 10 minutes, 25° C.). The supernatant was discarded, the pellet resuspended in 5 ml physiological salt solution and re-centrifuges. The last cycle was repeated 3 times, and the pellet was finally re-suspended in 5 ml physiological salt solution. Next, 630 μl lysozyme (obtained from Sigma, the Netherlands) solution (20 mg/ml) was added and the mixture was stored at 30° C. (1 hour) immediately followed by an additional incubation at room temperature (1 hour). The final solution was centrifuged (4000 rpm, 10 minutes); the supernatant (=cell lysate) was carefully removed and stored at −20° C.

### EXAMPLE 2

### Preparation of a Wheat Flour Matrix

A representative way to prepare a wheat flour matrix is as follows. A flour suspension was prepared using Kolibri flour (obtained from MENEBA, the Netherlands); 6.9 g flour was added to 11 g deionised water while mixing with a vortex. The flour suspension was homogenised with an Ultra-Turrax mixer and placed on a sinusoidal Coulter mixer (obtained from Coulter Electronics Limited, United Kingdom) for 10-30 minutes.

Immediately after its preparation, the flour suspension has to be incubated in a waterbath at 30° C. for at least 30 minutes with or without enzyme. The samples are then degassed using an ultrasonic waterbath (obtained from Sonicor Instrument Cooperation, USA). The wheat flour matrix is ready to be measured in the matrix screening.

### EXAMPLE 3

### Preparation of a Soy Derived Water-Unsoluble Substrate (WUS) Matrix

A representative way to prepare a soy derived WUS matrix is as follows. A 5% w/v soy WUS preparation was made in H₂O. Under continuous stirring the Soy-WUS was incubated overnight at room temperature. After sedimentation of the large particles the supernatant was collected and is ready for the use in the application screening.

### EXAMPLE 4

### Measurements of enzyme activity in milk matrix using Ultrasonic Spectroscopy

Ultrasound measurements of activity of three enzymes in milk were performed as follows. The ultrasonic spectrometer HR-US 102 was operated and set-up following the manufacturers instructions (Ultrasonic Scientific, Ireland).

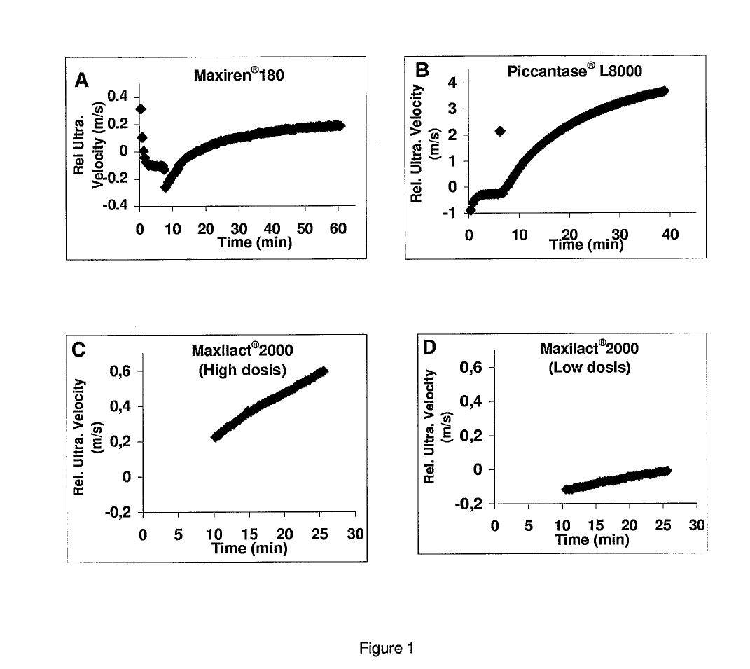

A 1 ml ultrasonic cell of an HR-US 102 ultrasonic spectrometer (Ultrasonic Scientific, Ireland) was filled with bovine milk and allowed to equilibrate at 30° C. After temperature equilibration, the enzyme was added and the course of the reaction was followed. Various enzymes were tested and additions were as follows:
A. Maxiren® 180 was diluted 1:100 and added to the milk (20 μl enzyme/1 ml milk)
B. Piccantase® R8000 was diluted 1:100 and added to the milk (30 μl enzyme/1 ml milk)
C. Maxilact® 2000 was diluted 1:100 and added to the milk (10 μl enzyme/1 ml milk)
D. Maxilact® 2000 was diluted 1:1000 and added to the milk (10 μl enzyme/1 ml milk)
   All enzymes were obtained from DSM Food Specialties, The Netherlands. The activity curves are shown in FIG. 1, graphs A to D.

The data show that the ultrasound technology is capable of measuring very diverse enzyme activities like protease (Maxiren® 180), lipase (Piccantase® R8000) and β-lactase (Maxilact® 2000) in one matrix (bovine milk). Thus, the Ultrasonic Spectroscopy methodology is very well suited as generic detection methodology in the matrix screening approach according to the invention. Also, the rate in signal change is dependent on the amount of added enzyme, as is clearly shown by the two Maxilact® curves (high and low doses). The graphs A and B for Maxiren® and Piccantase® R8000 also show the 8 minutes equilibration period.

### EXAMPLE 5

### Measurements of Enzyme Activity in Cheese Curd Matrix Using Ultrasonic Spectroscopy

Ultrasound measurements of activity of one enzyme in cheese curd prepared according,to Example 1 were performed as follows. The ultrasonic spectrometer HR-US 102 was operated and set-up following the manufacturers instructions (Ultrasonic Scientific, Ireland).

Cell 1 of the HR-US 102 ultrasonic spectrometer was filled with cheese curd+Piccantase® R8000 and Cell 2 of the HRUS was filled with cheese curd alone and the course of the reaction was followed for both cells at 30° C.

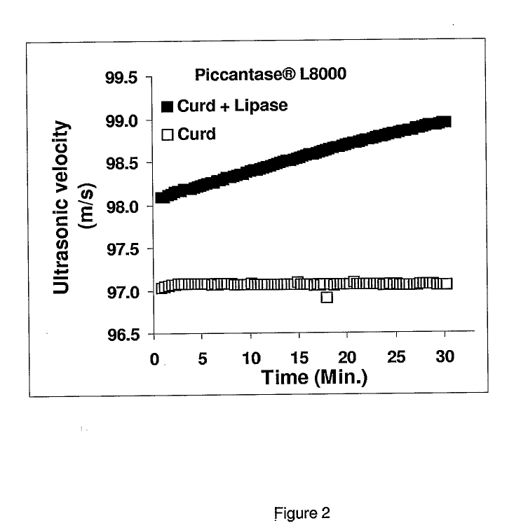

FIG. 2 shows that the ultrasound technology is capable of measuring enzyme activities like lipase (Piccantase® R8000) in cheese curd matrix.

### EXAMPLE 6

### Measurement of Enzyme Activity in Wheat Four Matrix Using Ultrasonic Spectroscopy

Ultrasound measurements of activity of five enzymes in wheat flour suspensions were performed as follows.

A flour suspension was prepared as described in Example 2. Subsequently, the flour suspension was incubated in a waterbath at 30° C. for at least 30 minutes with or without enzyme. The samples were degassed using a ultrasonic waterbath (obtained from Sonicor Instrument Cooperation, USA).

The ultrasonic spectrometer HR-US 102 was operated and set-up following the manufacturers instructions (Ultrasonic Scientific, Ireland). Cell 1 of the HR-US 102 was filled with a flour suspension+enzyme, and Cell 2 was filled with a flour suspension without enzyme. After an equilibration of the sample to 30° C. the ultrasonic velocity of the samples was measured according to manufacturers instructions.

Various enzymes were tested and additions were as follows:

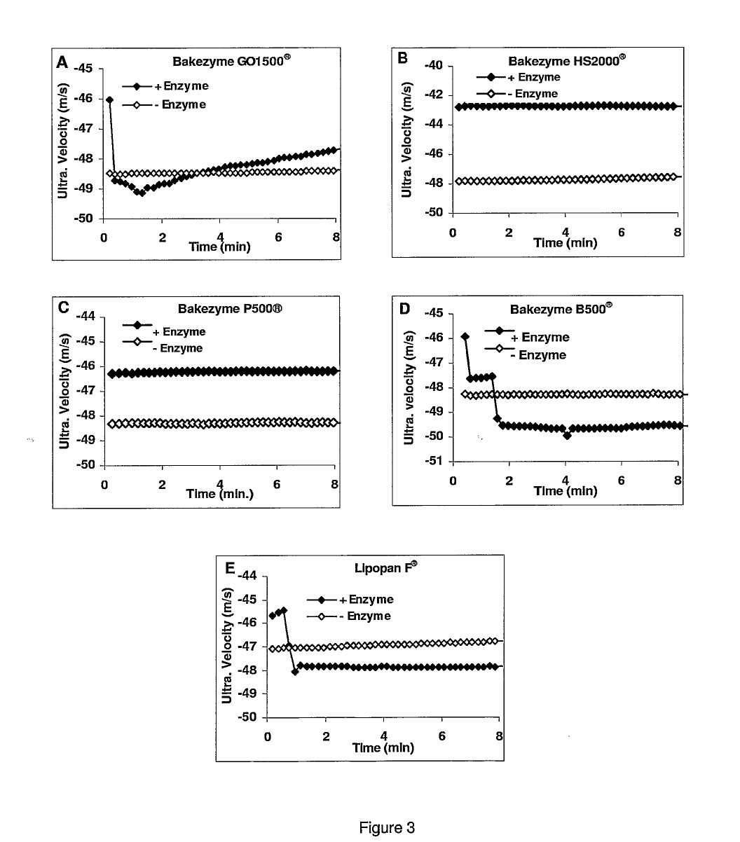

A. Flour suspension incubated with 500 ppm * Bakezyme GO1500®
B. Flour suspension incubated with 300 ppm * Bakezyme HS2000®
C. Flour suspension incubated with 38 ppm * Bakezyme P500®
D. Flour suspension incubated with 171 ppm * Bakezyme B500®
E. Flour suspension incubated with 60 ppm * Lipopan-F®
   Note: * ppm is mg enzyme/kg flour
   All Bakezyme enzymes were obtained from DSM Bakery Ingredient, The Netherlands, Lipopan-F® was obtained form Nolvozymes, Denmark).
   The activity curves are shown FIG. 3, graphs A to E. The data show that the ultrasound technology is capable of measuring very diverse enzyme activities like Glucose Oxidase (Bakezyme GO1500®), Xylanase (Bakezyme HS2000®), Amylase (Bakezyme P500®), Protease (Bakezyme B500®) and Lipase*(Lipopan F®) in a wheat flour matrix.

### EXAMPLE 7

### Measurements of Enzyme Activity in Soy-WUS Matrix Using Ultrasonic Spectroscopy

Ultrasound measurements of activity of two enzymes in soy-WUS prepared according to Example 3 were performed as follows. The ultrasonic spectrometer HR-US 102 was operated and set-up following the manufacturers instructions (Ultrasonic Scientific, Ireland).

Cell 1 of the HR-US 102 ultrasonic spectrometer was filled with soy-WUS+Enzyme and Cell 2 of the HRUS was filled with soy-WUS alone and the course of the reaction was followed by means of relative ultrasonic velocity measurements at 30° C.

The following enzymes were tested:

Pectinex® Ultra SP (Novozymes, Denmark) diluted 10 μl/500 ml WUS

Rapidase® Press (DSM Food Specialities, The Netherlands) diluted 50 μl/500 ml WUS.

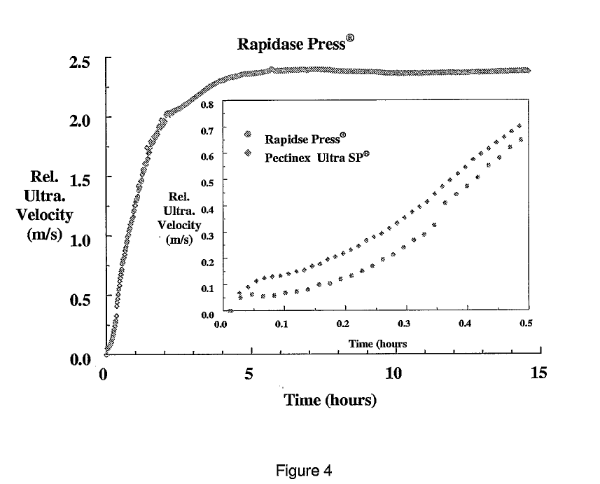

FIG. 4 shows that the ultrasound technology is capable of measuring enzyme activities like pectinase (Rapidase Press® and Pectinex ultra SP®) in a Soy-WUS matrix.

### EXAMPLE 8

### Measurement of Enzyme Activity, Spiked in Supernatant and Cell Free Extract in Milk Matrix Using Ultrasonic Spectroscopy

The activity of an enzyme spiked in Bacillus subtilis culture supernatant (A) and in a Bacillus subtilis cell free extract (B) was determined by Ultrasonic measurement. The Ultrasonic spectrometer HR-US102 was operated and set up following the manufacturers instructions (Ultrasonic Scientific, Ireland).

A 1 ml Ultrasonic cell of an HR-US102 Ultrasonic spectrometer was filled with bovine milk and allowed to equilibrate at 30° C. After temperature equilibration, 100 μl supernatant derived from a standard 48-hour B. subtilis fermentation (A) or 100 μl of a cell-free extracted derived from the same 48 hour B. subtilis fermentation (B) was added and the course of the reaction was followed. In both experiments the supernatant and the cell-free extract were analysed with or without spiking with a lactase. For spiking, Maxilact® 2000 was diluted 1:1000 in the B. subtilis samples.

The data show that the ultrasonic technology is capable of measuring enzyme activity in a complex protein preparation.

The supernatant and the cell free extract were prepared as follows:

A B. subtilis strain was inoculated directly from a freeze tube into 100 ml TY broth (16 gram trypton/l, 10 gram yeast extract/l en 5 gram NaCl/liter; pH adjusted to 7 with NaOH) and incubated at 37° C. and 250 rpm during 48 hours. After 48 hours of fermentation, the cells were harvested by centrifugation at 5000 rpm for 10 minutes. The supernatant was collected, filtrated using a disposable 0.22 μm steriflip vacuum system (Millipore, Bedford USA) and stored at −20° C.

The cell pellet was suspended in phosphate buffered saline solution (PBS), the amount of PBS used was corrected for the optical density of the 48 hour culture. The cell suspension was divided in 1 ml aliquots in tubes containing glass pearls for cell disruption. The disruption was performed in the Fastprep® (Q-Biogen, lllkirch, France) by using the Blue matrix protocol recommended by the supplier. The disruption was performed twice with sample precooling and cooling on ice in between. After the disruption cell debris and matrix were removed by centrifugation for 1 minute at 10.000 g. The cell free extract was store at −20° C.

1. A method for identifying an enzyme of interest comprising screening a set of candidate enzymes on an application matrix, preferably on at least a medium throughput level, and selecting enzymes for their ability to produce a functionality change in the application matrix.

2. A method according to claim 1 comprising
a) providing a set of candidate enzymes,
b) screening said set of candidate enzymes on an application matrix, preferably on at least a medium throughput level,
c) selecting a subset of enzymes that produces a functionality change in the application matrix,
d) selecting an enzyme from the subset of enzymes with a desired activity in a further application test,
e) defining the enzyme activity (ies) of the selected enzyme(s).

3. A method according to claim 2, wherein the functionality change of feature c) is a generic functionality change in the application matrix.

4. A method according to claim 2, wherein the functionality change of feature c) is a defined functionality change in the application matrix.

5. A method according to claim 2, wherein the functionality change of feature c) is measured using ultrasonic spectroscopy.

6. A method according to claim 2, further comprising repeating step c) to select a further subset of enzymes that produces a more stringent functionality change in the application matrix than the earlier measured functionality change of feature c).

7. A method according to claim 1, wherein the set of candidate enzymes is provided by a set of genes cloned and expressed in a suitable host organism, i.e. by an expression library.

8. A method according to claim 7, wherein the expression library is refined by removing redundant clones and/or by removing clones that have no or no significant protein over-expression.

9. A method according to claim 1, wherein the application matrix is full fat milk, curd, cheese slurry, bread dough, grain flour suspension, grain meal slurry or soybean meal slurry.

10. A method for the production of an enzyme selected by the method according to claim 1 comprising growing a microorganism capable of expressing said enzyme under conditions conducive to production of said enzyme and recovering the produced enzyme.

11. The method of claim 10, wherein the microorganism is a host organism transformed with a polynucleotide containing an expression cassette encoding the selected enzyme.

12. Use of a functionality change in an application matrix as a screening parameter for selecting desired enzyme activities.

13. Use of ultrasonic spectroscopy in the screening of enzyme activities.
